(19) United States
(12) Patent Application Publication
(10) Pub. No.: US 2021/0316029 A1
(43) Pub. Date: Oct. 14, 2021

(54) DEVICE AND METHOD FOR RECYCLING OF RESPIRATORY MASKS AND OTHER PERSONAL PROTECTIVE EQUIPMENT

(71) Applicant: University of Louisiana at Lafayette, Lafayette, LA (US)

(72) Inventors: Mark E. Zappi, Lafayette, LA (US); William Holmes, Lafayette, LA (US); Andrei Chistoserdov, Lafayette, LA (US); Wayne Sharp, Lafayette, LA (US); Alex Zappi, Lafayette, LA (US); Rafael Hernandez, Lafayette, LA (US); William Chirdon, Lafayette, LA (US)

(21) Appl. No.: 17/347,184

(22) Filed: Jun. 14, 2021

Related U.S. Application Data
(63) Continuation-in-part of application No. 17/209,586, filed on Mar. 23, 2021.
(60) Provisional application No. 62/993,197, filed on Mar. 23, 2020.

(51) Int. Cl. A61L 2/26; A61L 2/20; A61L 2/10

(52) U.S. Cl. CPC A61L 2/26; A61L 2/202; A61L 2/208; A61L 2202/24; A61L 2202/121; A61L 2202/11; A61L 2/10

(57) ABSTRACT

A method and device for treating contaminated or potentially contaminated items is provided. The method will include the steps of placing the items in a reactor. The reactor is then sealed and ozone or ozonated gas is sent into the reactor. The items are held in the reactor long enough to complete the decontamination process. The reactor is then opened. Next there is a period of passive reaction so that the ozone can be degraded from the pores and surfaces of the items. The device provided herein includes a reactor, an ozone generator, a rack for holding said items; and a monitor for determining the level of ozone in the reactor.

### CROSS REFERENCE TO RELATED APPLICATIONS

This is a continuation in part of U.S. Non-Provisional application Ser. No. 17/209,586, titled Device and Method for Recycling of Respiratory Masks and Other Personal Protective Equipment” filed on Mar. 23, 2021, which claims priority to “U.S. Provisional Application No. 62/993,197, titled “Device and Method for Recycling of Respiratory Masks and Other Personal Protective Equipment” filed on Mar. 23, 2020.

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

Not Applicable.

### DESCRIPTION OF THE DRAWINGS

The drawings constitute a part of this specification and include exemplary embodiments of the Device and Method for Recycling of Respiratory Masks and Other Personal Protective Equipment, which may be embodied in various forms. It is to be understood that in some instances, various aspects of the invention may be shown exaggerated or enlarged to facilitate an understanding of the invention. Therefore, the drawings may not be to scale.

FIG. 1 is a schematic depicting one embodiment of the Device and Method.

FIG. 2 is a schematic depicting one embodiment of the Device and Method wherein a wet tubular disinfection reactor is used with additional oxidizer inputs along the length of the reactor.

FIG. 3 is a schematic depicting one embodiment of the Device and Method wherein a semi-wetted tubular disinfection reactor is used.

FIG. 4 is a schematic depicting one embodiment of the Device and Method wherein a semi-wetted, ultraviolet irradiated u-shaped tubular disinfection reactor is used.

### BACKGROUND

Medical personnel, first responders, and other personnel rely on Personal Protective Equipment to protect them from infection when performing their jobs. Personal Protective Equipment (PPE) may include respiratory masks, hoods, gowns, disposable scrub sets, HazMat (Hazardous Material) suits, or other items that serve as a barrier against viruses, bacteria, fungi, or other organisms and substances, such as blood, urine, and saliva, that can cause illness. Adequate supplies of PPE will be critical when a particular region is faced with an epidemic or pandemic. Normally, used PPE items are thrown out as waste and cannot be reused. While normal levels of PPE supplies may initially be adequate in the face of an outbreak of communicable diseases, as the pandemic spreads those supplies will likely be exhausted.

The invention described herein provides a device and method for treating used PPE items so that they can be used again. While the description and claims described herein focus on PPE items, those skilled in the art will find other applications for the device and method. For example, the invention may be applied for treatment of bed linens, surgical items, or any other materials that may have been exposed to virus particles or other pathogens.

### DETAILED DESCRIPTION

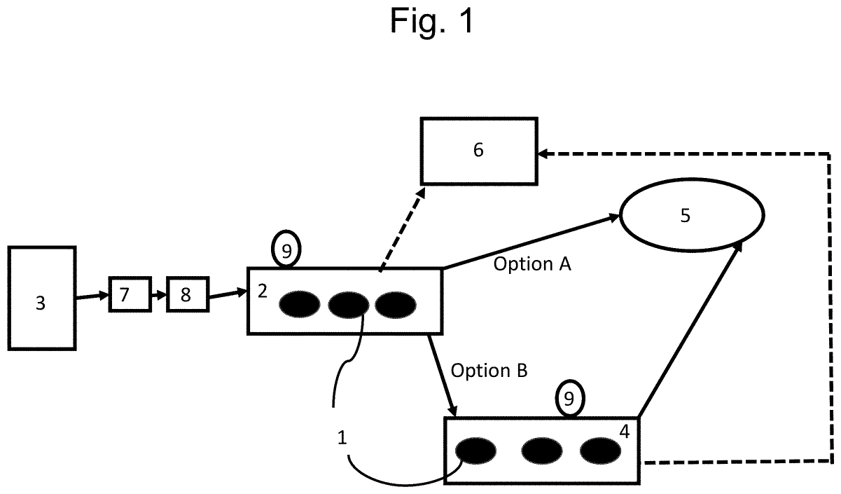

This device will utilize ozone gas (0₃) as the primary active agent for the disinfection method. With reference to FIG. 1, contaminated or potentially contaminated PPE items 1 are placed within reactor 2. PPE items 1 may include respirators (ex. N95), protective suits, goggles, and other personal PPE items. After PPE items 1 are placed within reactor 2, reactor 2 is sealed and ozonated air and/or ozonated oxygen is gassed into the sealed reactor. The sealed reactors that allows continuous flow-through of the ozonated gases or closed operations, thus ceasing the flow to provide for static treatment. Both continuous and static oxidation/disinfection modes may be applied.

The ozonated gas is kept in the sealed reactor long enough so that ozonated gas has sufficient time to interact completely with the potentially contaminated surfaces of the objects to be disinfected. For most disinfection applications the surfaces of typical PPE items will require only seconds to minutes of treatment in the reactor. The continuous flowing operation or mode allows enough gas contact time within the reactor to disinfect the media.

The ozonated gas is generated on-site using a portable or fixed ozone generator 3 (commercial or lab grade) using air and/or oxygen as the oxygen source. One type of ozone generator known to those skilled in the art is the Corona Discharge ozone generator. However, any other type of ozone generator known to those skilled in the art will suffice. Ozone generator 3 will be plumbed so that the ozonated gas is forced into the reactor containing the potentially contaminated/infected PPE. In one embodiment, ozonated gas will be pass through flowmeter 7 and mass totalizer 8 that are place intermediate ozone generator 3 and reactor 2.

After sufficient time is allowed for the ozone to disinfect the pathogen via static operation or a sufficient fluid residence time; under Option B the gas may be released into secondary reactor 4 that can serve as a pretreatment step, thus saving ozone. In an alternative embodiment (Option A), the used ozonated gas is passed to ozone destruction unit 5 to purge the carrier gas of any ozone (ozone is considered a pollutant).

Once the gas pressure within the primary reactor 2 is equalized to atmospheric pressure, then a period of passive reaction can be performed to allow the PPE to degrade the ozone from within its pores and/or surfaces. Because ozone is not a stable gas it will likely have to be generated on-site. The unstable character of ozonated gas is important to the method because this characteristic will allow passive removal of the ozone from the PPE. Alternatively, air may be passed after oxidation to allow for purging of the ozonated gases out of the reactor and inner pore volumes/spaces of the media being treated.

Pressures in reactor 2 and secondary reactor 4 may be monitored using pressure gauges 9.

This process can be utilized at a small, single PPE scale (e.g. a single N95 mask) or scaled to very large application (several HazMat suits and/or many PPE masks). Additionally, the device and method may be used for decontamination of medical devices, medical instruments, lab equipment, emergency equipment, or any other item needed decontamination.

The ozone gas contact and treatment device and method described herein disinfects the PPE items of pathogens and other hazards, including but not limited to viruses (including the corona virus or COVID 19 virus), bacteria, and fungi.

In a preferred embodiment, reactor 2 comprises a tubular reaction/disinfection reactor. The reactor may be straight or u-shaped, or any suitable shape to fit the environment constraints.

In a particularly preferred embodiment, the ozonated gas ozone concentrations going into the reactor and out are continuously monitored using an ozone monitoring system. In one embodiment the ozone monitoring system will comprise real-time gas ozone gas analyzer 6 with sample points at ozone generator 3 and along the length of the reactor tube to ensure that appropriate levels of ozone are present. The device can be configured so that ozonated gas can be introduced into either end of reactor 2 or run only from one direction.

In one or more embodiments, baffles are installed within the reactor to facilitate improved gas mixing. In one or more embodiments, the diameter or reactor 2 is varied over the length of the reactor 2 to minimize “wasted” or “dead” reactor volume.

Multiple secondary reactors 4 can be plumbed in series to fully use the ozone already generated so that as ozonated gas exits the primary disinfection reactor, that gas can be passed through a secondary or tertiary reactor system which fully uses the generated ozone.

In one embodiment, the disinfection reactors can also be fully charged with ozonated gas and run as true batch systems such that the reactor is fully charged with ozonated gas and the reactions allowed to occur until some C−T dose (concentration−contact time=C−T) is reached. In another embodiment, a wire rack is used to hold the masks upright and the cupped part of a concave mask is facing the incoming gas stream to maximize gas contact with the pores of the materials being treated.

Operation of the reactor can be in batch, semi-batch, and/or continuous modes of operation. The reactor can be operated within a positive or negative pressure operation via introduction of the gases or removal. A push/pull operation of gas surging can also be established within the reactor to facilitate maximum contact of the oxidizers with all surfaces of the object being decontaminated/disinfected. The disinfection agents may be introduced at either said of the reactor or both or added along the length of the reactor.

The reactor may be operated with a period of static disinfection time with no more disinfection agents being added, then purged with air to remove residual disinfection agents or immediately after disinfection treatment, the flowing gas switched to air or pure oxygen to sweep the reactor of residual disinfection agents.

In one or more embodiments, the reactor may be operated with no disinfection liquids, misted/sprayed disinfection agents, or fully filled (saturated) with disinfection agents or a combination of one or two or three of these options

In one or more embodiments, an ultra-violet or “UV” lamp can be inserted into the reactor to provide secondary disinfection in conjunction with ozone, thus producing hydroxyl radicals.

In another embodiment, The PPE items laid out on the reactor support rack can be removed easily from the reactor, then inserted into reactor 2 with only compressed air and/or nitrogen and/or carbon dioxide and/or any other gas to assist in the removal of the residual ozone from the PPE.

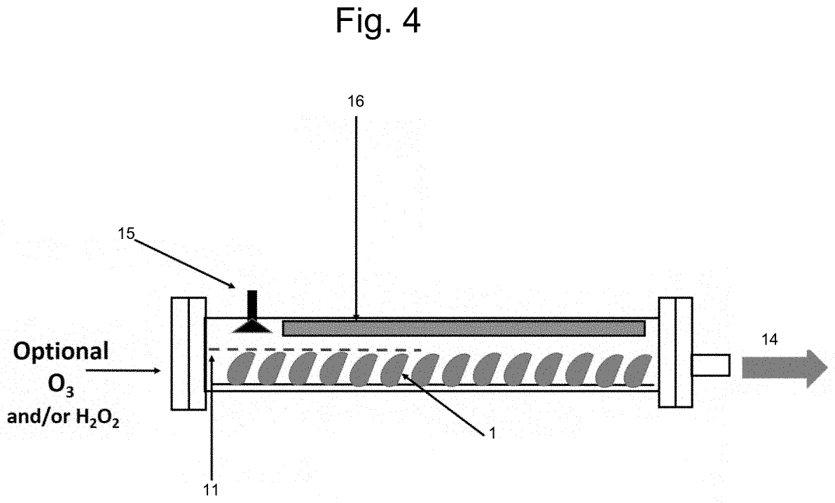

FIG. 4 shows a further embodiment, in which UV lamps 16 can be inserted into the de-ozonation reactor 2 to assist and accelerate the dissipation of ozone residuals. The residual ozone within the ozonated gas may be removed from the final exiting gas stream using a commercial ozone destruction system.

FIG. 4 also shows a water line 11 that can be used for wetted or semi-wetted applications.

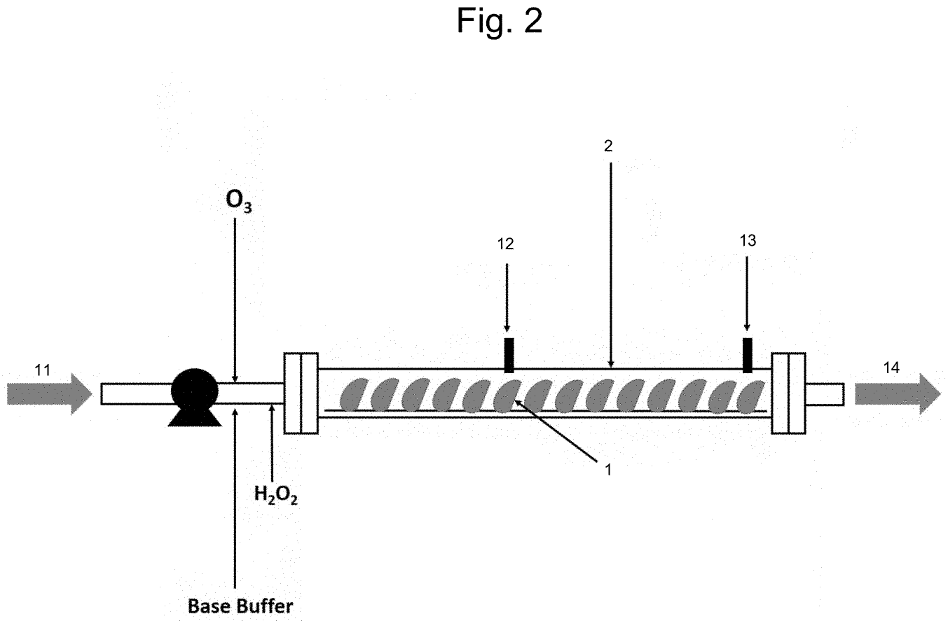

In another embodiment, the primary and secondary reactors can be filled with ozonated water to use a wet disinfection mode within the reactors. FIG. 2 shows such an embodiment. Clean potable water enters the system at 11 through a pump. Ozone may then be introduced via a venture injector and/or offline sparge tank. This adds highly ozonated water to the reactor inflow. Hydrogen peroxide injection using a chemical dosing pump to desired levels and/or a weak base to pH-auto-decompose oxygen into hydroxyl radicals may also be introduced, as shown in FIG. 2.

FIG. 2 also shows measurement devices to gauge the pH level and ozone and/or H₂O₂ levels within the reactor. If needed, spent water is disinfected via ozone, ultra-violet, and or. H₂O₂to remove any residual pathogens and then drained or recycled at 14 in FIG. 2.

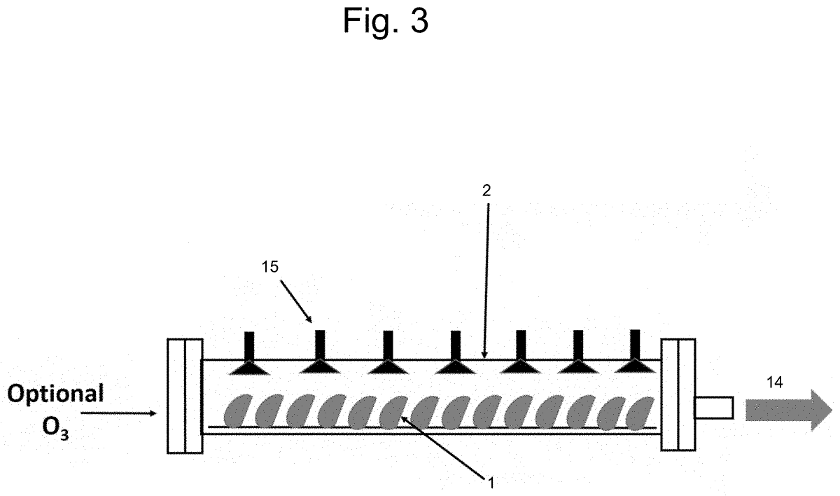

However, for many PPE items (e.g. N95 masks), wet disinfection will not be suitable. In another embodiment, hydrogen peroxide can be batch added or continuously added along with ozonation to initiate peroxone reactions to produce large amounts of the hydroxyl radical, which is a much more effective disinfection agent than ozone alone. FIG. 3 shows a semi-wetted option in which a plurality of hydrogen peroxide aspirators 15 are used.

Hydrogen peroxide can also be added without ozone but with the addition of iron salts to use Fenton's Reaction within the reactors to produce the hydroxyl radical.

In another embodiment, the reactor can be configured so that it resembles a sealed hanging clothes closet/box to allow for disinfection of hanging PPE. The rack can be modified to hold open gloves set into the reactor for disinfection. It will be obvious to those skilled in the art to disinfect other medical devices using both reactor designs—those using ozone alone those using hydrogen peroxide and UV light alone or in combination with ozone.

In another embodiment, in hydrogen peroxide embodiments, the solution pH in the reactor may be increased to allow for ozone to be converted into the hydroxyl radical. The disinfection system can be fully automated using controls and solenoids. An ozone monitor can be installed onto the exit ports of the deozonation reactor to ensure no residual ozone is within the exit gas.

In another embodiment, the temperature of disinfection and deozonation within the reactors can be elevated to enhance the rate of reaction. This can be accomplished using a variety of heating options, including but not limited to heat wrapping, annular tubed reactor, pre-reactor heat exchanger, and other options known to those skilled in the art. Baffles can be added into both the disinfection and deozonation reactors to increase the mixing of the gases within the reactors.

Vacuum may be applied prior to the injection of either the ozonated gas for the disinfection reactor or the sweep gas within the deozonation reactor to enhance the gas entrance into the small pores of the PPE. A variety of reactor configurations may be used, such as a box or some other more dimensionally equal reactor, not just tube reactors.

An in-line section of vortexing fins can be added into the reactors. The ozonated gas and/or liquids can be pulsed and/or oscillated within the reactors to improve pore access of the disinfection agents. Ultrasound can be added to the reactors to enhance mass transfer and completion of the reactions.

In another embodiment, ozonated gas may be directed injected into the PPE within an encased vessel using a directed gas spray—similar to a sand blasting box or glove box.

A pressure-drop in-line vortex or venture tube gas introduction system can be used to introduce liquid reactants into the reactors. Mass flow totalizers and pressure gauges can be added before, along, and after the reactors to monitor process conditions. Fans or some similar mixing and velocity accelerators may added to the reactors to enhance mass transfer. The ozonated gas and/or the deozonation gas does not have to be introduced at the ends of the reactor—the gas can be introduced anywhere along the length of the reactors or any combination of several injection points on the reactors.

The disinfection nature of the proposed invention is not only limited to ozone but also oxidizer radicals, hydrogen peroxide, UV photons, heat, and/or any other disinfection mechanism(s).

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies.

1. A method of treating contaminated or potentially contaminated items in a reactor, comprising:
a. Placing said items in a reactor;
b. Sealing said reactor;
c. Gassing ozone gas or ozonated gas into said reactor in a continuous manner;
d. Keeping said items in reactor long enough so that ozone gas or ozonated gas has sufficient time to interact completely with the potentially contaminated surfaces of said items;
e. Unsealing said reactor such that gas pressure within said reactor is equalized to atmospheric pressure;
f. Allowing a period of passive reaction so that said items can degrade the ozone from within their pores and surfaces; and
g. Purging said reactor with inert gas to remove any residual infection agent.

2. A reactor system comprising:
a. a reactor shaped so that when ozonation gases are passed through said reactor, the flow of said ozonation gases is continuous through said reactor; and
b. a secondary disinfectant means.

3. The reactor system of claim 2 wherein said secondary disinfectant means consists of at least one of the following: hydrogen peroxide mist and at least one ultraviolet lamp.

4. The reactor system of claim 2 wherein said reactor is tubular and has a diameter and a volume, wherein said dimeter varies along the length of said reactor so that unused reactor volume is minimized when said ozonation gases are passed through said reactor.

5. The reactor of claim 2 wherein said secondary disinfectant means comprises a water line.

6. A method of treating contaminated or potentially contaminated items in a reactor, comprising:
a. Placing said items in a reactor, said reactor having a sealable opening and a rack for holding said items;
b. Sealing said reactor at said sealable opening;
c. Gassing ozone gas or ozonated gas into said reactor;
wherein said gassing step may is performed in a batch, semi-batch, or continuous mode as selected by a user.

7. The method of claim 6 wherein said reactor can be operated in a positive or negative pressure.

8. The method of claim 6 further comprising the step of aspirating peroxide into said reactor.
